(19) United States
(12) Patent Application Publication
(10) Pub. No.: US 2005/0211562 A1
(43) Pub. Date: Sep. 29, 2005

(54) Method for coating joint surfaces of metals used to form prostheses

(76) Inventors: Thomas G. Rowe, Rock Creek, OH (US); Harry E. Corl III, Twinsburg, OH (US)

Correspondence Address: NAWROCKI, ROONEY & SIVERTSON;SUITE 401, BROADWAY PLACE EAST, 3433 BROADWAY STREET NORTHEAST, MINNEAPOLIS, MN, 554133009, US

(21) Appl. No.: 11/001,360

(22) Filed: Dec. 1, 2004

Related U.S. Application Data
(63) Continuation-in-part of application No. 10/435,813, filed on May 12, 2003, now abandoned.
Application No. 10/435,813 is a continuation-in-part of application No. 09/859,352, filed on May 17, 2001, now abandoned.

(51) Int. Cl. C25D 5/34; A61F 2/32

(52) U.S. Cl. 205/219; 623/22.43

(57) ABSTRACT

A process electroplates a metal workpiece with thin dense chromium. The workpiece is first activated by submerging in an aqueous solution of sulfuric acid and a biflouride salt, preferably ammonium biflouride. The biflouride salt reacts to form HF. A preferred activating bath has a 35% sulfuric acid concentration of 4 ounces per gallon of ammonium biflouride salts. A positive DC voltage is applied between the workpiece and a cathode in the bath. The workpiece is then submerged in a chromium plating bath formed of chromic acid sulfate to produce the chromium plate. A DC plating voltage initially 3 volts produces a current flow of about 1.5-2.5 amps per square inch of workpiece area. In a preferred process, the plating voltage is continuously increased to an eventual value of about 4.5 v. The process is particularly suited to plating cobalt-chromium alloys.

This is a continuation-in-part application filed under 35 U.S.C. §1.53(b)(2) claiming priority under 35 U.S.C. §120, of U.S. patent application Ser. No. 10/435,813 having a filing date of May 12, 2003 and filed under 35 U.S.C. §1.53, which is a continuation-in-part of the application filed under 35 U.S.C. §1.53(b)(2) claiming priority under 35 U.S.C. §120, of U.S. patent application Ser. No. 09/859,352 having a filing date of May 17, 2001, filed under 35 U.S.C. §1.53(b).

### TECHNICAL FIELD

The present invention relates generally to the field of plating and in particular, to a process for forming a hard, adherent, chrome plating. The invention has particular use in forming a superior bearing surface in articulated prosthetic joints. The process is useful in forming a chrome plate that is extremely hard, dense, and adherent, and that has a low friction, non-magnetic outer surface susceptible to little corrosion, wear damage, etc. The process is successful in plating cobalt-chrome alloys, and may also succeed in plating other metals such as stainless steel.

### BACKGROUND OF THE INVENTION

Movable joints have been utilized in many different technical areas, from medical implants to automobile parts, with each technical area having different, important characteristics. In some applications, the amount of constant load that a joint can maintain over a long duration is important. In other applications, the maximum load that a joint can support over a short period of time may be important. In still other applications, the wear resistance of the joint when the parts of the joint are in relatively constant movement is important. Most applications require a mix of these important factors.

One such application is the use of a ball-type joint to replace a natural joint in a human or animal. Ball joints have proven useful in this application because, like the natural joint that the implant is replacing, the joint provides a wide range of motion. However, under these conditions, it is important to have a joint suited to relatively constant motion under differing loads without becoming worn and requiring replacement. Joint replacement requires invasive surgery, so a durable joint lessens the risk from such surgery and possible resulting complications.

The present invention is helpful in producing any movable joint, but is particularly applicable to ball-type joints. A movable ball joint is typically comprised of two main parts; a spherical ball portion and a socket portion. The socket design encloses more than half of the ball portion, thereby retaining the ball portion and allowing universal joint movement with respect to the socket.

Traditionally, both the ball and socket have been made from the same material. For example, in the field of medical implants, the most commonly utilized material has been cobalt-chromium alloys. These materials are used for this purpose because they are strong enough to withstand the day-to-day forces applied to them and they are light enough to be suitable as a replacement for the natural joint, and have other advantages as well. However, the wear between the two parts has made the life of previous designs of these devices shorter than desirable.

One proposed solution has been to use different materials to construct the each of the joint parts, wherein one material is tougher and harder than the other material. This allows the replacement of a single part instead of the replacement of both parts. However, invasive surgery is still necessary to remove and replace the one part when it becomes worn, so this solution is not optimal.

A further problem with dissimilar joint component material designs is that one component accelerates the wear experienced by the other. Corrosion and wear on the one releases metal ions from the implant components into adjacent body tissue. More often than not, these ions are incompatible with the body, and can thus lead to physical reactions such as for example, inflammation, bone degeneration, and healing disturbances.

If either one of the implant components degrades, friction between the two components increases, so that the joint does not operate smoothly. Corrosion and wear damage contributes to a decrease in both the static and the dynamic strength and stability of the implant. This deterioration has been well-documented in hip joint prostheses.

Hip joint prostheses typically have a ball joint design that includes a cup-shaped bearing portion, called the acetabular cup, and a mating portion, which is typically a spherical ball element, called the head. The head articulates in the cavity of the cup to permit motion. In a full replacement hip joint prosthesis, the head is provided by removing the existing femur ball, and implanting a prosthetic head with a rod-like member referred to as the neck and stem which is anchored to the femur. In another design, known as a surface replacement prosthesis, the head is provided by resurfacing the existing femur ball with a covering, typically metal.

The socket of the acetabular cup is typically defined by a layer of ultra-high molecular weight polyethylene polymer (UHMWPE). The useful lifetime of the prosthesis depends for the most part on wear of this polyethylene cup (i.e., the UHMWPE). One cause of wear on the cup is abrasion caused by the motion of the head. This abrasion breaks fine particles from the cup. The particles migrate into the surrounding tissues and initiate biological processes such as swelling and soreness that ultimately lead to failure of the prosthesis.

While discussions to this point have focused on movable joints such as hip joint prostheses, any component that may degrade due to corrosion, abrasion, wear, etc. due to environmental conditions or interaction with other components of a system may profit from this invention.

### SUMMARY OF THE INVENTION

The present invention offers a solution to friction, corrosion, and wear in a prosthetic joint by providing a hard, adherent chromium layer forming the bearing surface of the joint. This chromium surface reduces wear between the joint surfaces, such as both the ball and socket portions of a ball joint, by virtue of its intrinsic hardness and lubricity. The surface produced by the invention is particularly compatible with a socket made from UHMWPE.

One version of the invention is a process for preparing a surface for electrolytically plating such a hard, adherent chromium layer thereon. The process includes first providing a workpiece formed of an alloy comprised at least in part of cobalt and chromium and that carries the surface to be prepared. Then at least a selected area of the surface is activated by submerging the selected area in an aqueous solution including sulfuric acid and a dissolved biflouride salt. In a preferred version the biflouride salt comprises ammonium biflouride. The biflouride salt reacts with the sulfuric acid to form hydrofluoric acid in the activating bath.

The activating step usually further comprises applying a positive current between the workpiece and a cathode in the activating solution.

A further version of the invention applies a chrome plate to the activated workpiece surface by submerging the selected area as treated by the activating step in a chromic acid sulfate plating bath. Then chromium is plated on the selected area by applying a negative DC plating voltage between the workpiece and an anode in the plating bath.

A number of detailed features of these steps improve the resulting chrome plate. Applying a positive DC voltage between the workpiece and a cathode in the aqueous solution improved the activating step. Particular voltage levels and concentrations of the biflouride salt and the sulfuric acid optimize the activation process.

At this time a preferred activating step uses an aqueous solution of sulfuric acid with a concentration of around 35% and ammonium biflouride crystals added at a concentration of around 2 to 6 oz. per gal., coupled with a DC voltage that produces a current density of about 1 to 4 amp. per sq. in. of selected area seem to be optimal.

The plating step also has preferred combinations of negative DC voltage levels, voltage profiles, and chromic acid sulfate concentrations. A particular combination of parameters that provides good results has an initial current density of from 1 to 4 amp. per sq. in. of selected surface. The voltage producing this current is then incremented by about 0.1 v. about every 10 sec. until the voltage reaches about 4.5 v. This voltage level and voltage profile produces a dense, hard, smooth, adherent chrome plate suitable for use as a bearing surface in a prosthetic joint such as a hip joint.

This plating process can be used to form a hip joint generally having a first portion and a second portion with either the first portion or the second portion having the chromium outer surface produced by this process. For example, one embodiment of the present invention generally provides a ball joint, having a ball portion comprising at least a deposition of chromium forming an outer surface of the ball portion. Alternatively, the socket portion may have a deposition of chromium forming an interface surface thereon.

In use, the ball portion is adapted for capture by and multi-axis rotation within a defined area of the socket portion. In each embodiment, the chromium deposition forms an interface surface between the first and second portions.

In a particular embodiment, the chromium material utilized for deposition on either the first or second portion of a movable joint is comprised of hexavalent chromium. The chromium material may be in the form of an electro-chemically bound, thin deposit of chromium on the outer surface of the portion such as created by this invention. In such an embodiment, the substrate may be comprised of a cobalt-chromium based alloy.

Furthermore, the chromium may be bonded to the outer surface of the portion by electro-deposition.

In a ball-type joint, the socket portion generally has an area constructed and arranged to receive the ball portion in a movable relationship within the confines of the defined area. In one embodiment, the socket portion of the joint is formed from ultra high molecular weight polyethylene. This material provides a suitable and complimentary surface to that of a chromium deposited ball portion, thereby providing increased wear resistance to the device.

The features provided above may be combined to provide an embodiment comprising a joint having a first portion, formed of a cobalt-chromium based alloy, with an outer surface coated with a chromium deposition applied over its outer surface, and a second portion formed from an ultra high molecular weight polyethylene material.

One application that joints, constructed according to the present invention, are particularly suited for is use in replacement of natural human or animal joints, such as knee, ankle, elbow, shoulder, spine, etc. However, the devices may be useful in any medical or non-medical application that, among other criteria, requires a joint with good wear resistance. Joints fabricated according to the present invention are also suited for these applications because they provide a reduction in fretting. Fretting is the production of wear debris through the interaction between two or more parts. The reduction of fretting within a living body reduces the chance of osteolysis, which occurs when wear debris enters the bloodstream.

One preferred method of producing a coated ball joint, comprises the steps of: providing a socket portion having an area adapted to receive a ball portion of the ball joint and the forming of either the ball or the socket portion having at least an outer interface surface comprised of chromium, wherein the ball portion is adapted to be received and captured, such that the ball portion is capable of rotatable movement, within an area of the socket portion. The method may also include the step of capturing the ball portion within the area of the socket portion. In a ball-type joint, wherein the ball is the first portion and the socket is the second portion, the socket has an area constructed and arranged to receive the ball in movable relation within the confines of the defined area and the ball portion adapted to be rotatably captured within the defined area of the socket portion.

As previously mentioned, one process of electroplating a metal workpiece with thin dense chromium, includes in detail, the steps of submerging an area of the metal workpiece in a 35% sulfuric acid solution having about 4 ounces per gallon HF as ammonium biflouride salts, and subsequently submerging the metal workpiece surface in a chromium plating bath. An initial negative DC voltage of about 3 volts is then applied between the workpiece, which serves as a cathode, and an anode in the plating bath. A suitable amperage for the voltage to provide is about 1.5-2.5 amps per square inch of cathode area.

The aforementioned benefits and other benefits including specific features of the invention will become clear from the following description by reference to the accompanying drawings.

### BRIEF DESCRIPTION OF THE DRAWINGS

FIG. 1 is a cut-away side view of a ball-type embodiment of the present invention wherein the socket has been attached to the bone surface of a patient.

FIG. 2 is a magnified cut-away side view of a portion of the ball of the implant of the embodiment of FIG. 1 showing the interface of chromium applied to the surface of the ball portion.

FIG. 3 is a cut-away side view of an embodiment of the present invention showing an interface of chromium applied to the surface of the socket portion.

FIG. 4 is a cut-away side view of the embodiment of FIG. 3.

FIG. 5 is a cut-away side view of an embodiment of the present invention in assembled condition showing the interface of chromium applied to the surface of the ball portion.

FIGS. 6-8 show a frame for supporting a workpiece during the activating and plating.

### DETAILED DESCRIPTION OF THE INVENTION

Referring now to the drawings wherein like reference numerals denote like elements throughout the several views. The invention teaches a plating process hereafter referred to as the process invention. The chromium plate formed by the process invention will be referred to as the process plate hereafter.

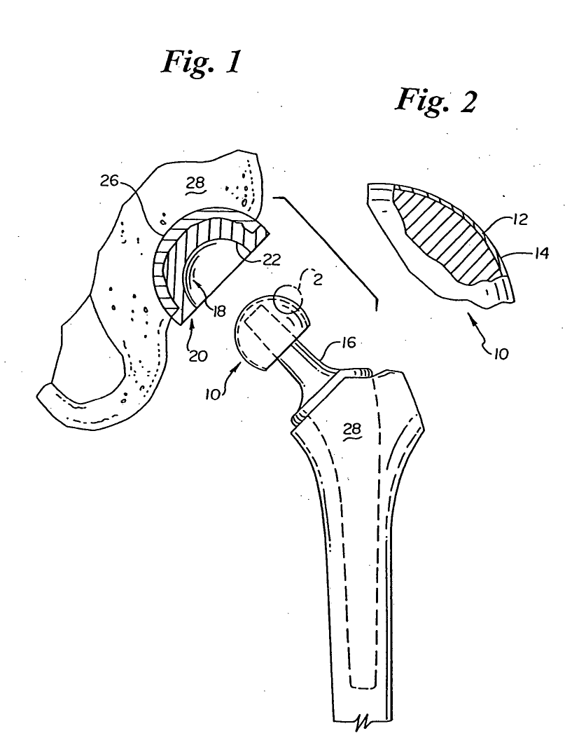

FIGS. 1 and 2 illustrate a side section view of a spherical prosthetic ball 10 that has the process plate forming the outer surface 12. Ball 10 serves as the ball portion of a hip prosthesis. Ball 10 fits in a socket portion 20 of the prosthesis. Ball 10 has a size and shape to match and engage the internal shape of a cup 18 formed in the socket portion 20. Cup 18 has an internal bearing surface 22 against which the ball portion 10 presses and moves.

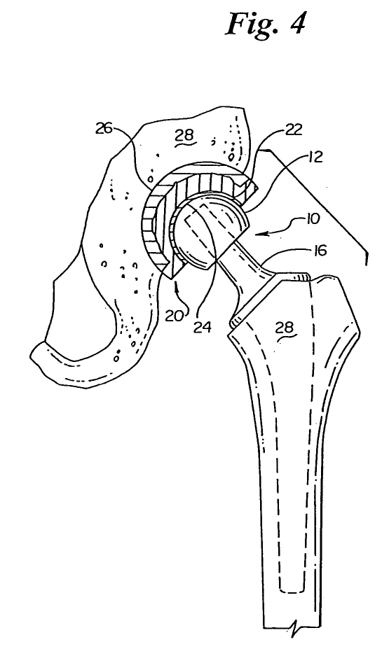

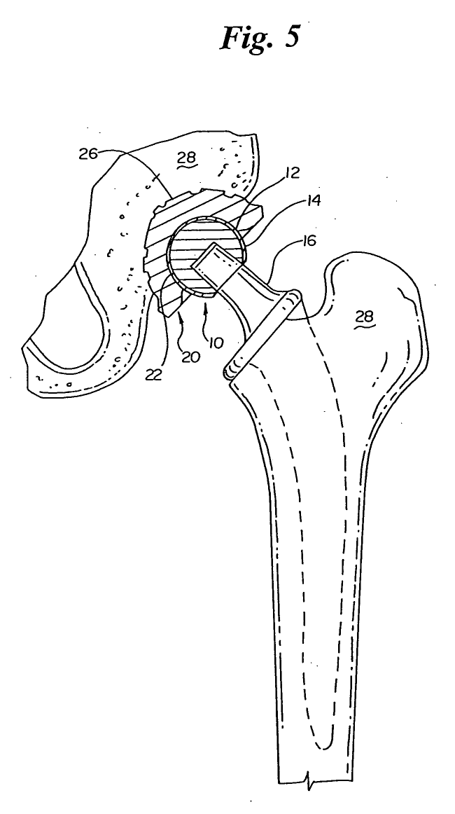

FIGS. 4 and 5 show cup 18 shaped to hold the ball portion 10 within the confines of the cup 18 and to allow the ball portion 10 to rotate within the confines of the cup 18. The ball portion 10 is typically attached to a stem 16 that can rotate relative to the socket portion 20 because of the rotatable engagement of the ball portion 10 with the socket portion 20.

The socket portion 20 and stem 16 of the ball portion 10 may be attached to an attachment surface such as femur 28 by any means known in the art. Some suitable examples of attachment means include: mechanical attachment assemblies, such as screws and nuts and bolts, and adhesive mechanisms, such as cement and glue, for example.

Furthermore, the shape of the surface 26 of the socket portion 20 utilized for attachment to the attachment surface 28 may be of any suitable shape known in the art. For example, FIGS. 1, 3, and 4 illustrate a socket surface 26 having a substantially uniform circular surface, whereas FIG. 5 illustrates a socket portion 20 having a non-uniform surface 26.

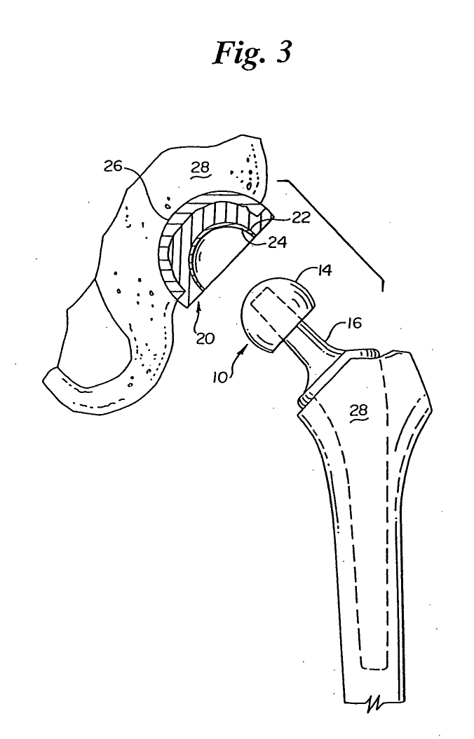

Either the outer surface 12 of ball 12 or the bearing surface 22 of the socket portion 20 may be coated with the process plate. In the embodiment shown in FIGS. 1, 2, and 5 the process plate is applied to the outer surface 12 of the first portion 10. In the embodiment shown in FIGS. 3 and 4, the process plate is applied to the outer surface 22, generally formed within the cup 18, of the second portion 20.

The one of the ball 10 and cup 18 to receive the process plate preferably is a cobalt-chromium alloy having the following approximate proportions by weight: 62% cobalt, 29.5% chromium, and 6.5% molybdenum. This alloy is commonly used in prostheses. The process invention deposits a superior process plate on such an alloy. Testing has confirmed the process plate provides superior compatibility with a UHMWPE mating surface.

A preferred joint has process plate on one of ball 10 and cup 18 to provide an interface with the material forming the other of the ball 10 and the cup 18. UHMWPE preferably forms the surface not covered with the process plate.

The process invention preferably uses electro-deposition. One suitable thickness for the process plate is approximately 2/10,000 of an inch, however the deposition may be as small as 50/millionths of an inch. The process of applying the coating may also include pre- and post-plating mechanical polishing.

The process plate is extremely hard and dense, low-friction chromium. The process plate formed by the process invention is a smooth, fine grained deposit that is uniform in thickness and appearance. The surface is free of blisters, pits, nodules and porosity, with minimal edge buildup.

The coating of the subject invention is uniformly deposited on metal workpieces or substrates. Generally, the coating is applied directly to the metal forming the ball 10 or cup 18 without an intermediate coating. It is preferably applied following completion of all previous processing, including, but not limited to, machining, brazing, welding, heat treating, and stress relieving.

The process plate thickness is 0.000025 inches-0.0006 inches (0.64 microns-15.38 microns). Depending upon the thickness specified, and the requirements for the particular use, the following thickness tolerance can be maintained: ±0.000010 inch to ±0.000050 inch (±0.25 microns to ±1.28 microns).

Articles having the process plate deposited on their surface can be repeatedly bent and twisted without the process plate chipping, flaking, or otherwise separating from the article surface. Articles coated with the process plate show no evidence of discoloration, cracking, flaking, rust or other change following repeated autoclave exposures.

Finally, as to biocompatibility, the process plate meets or exceeds USP Class VI Certification.

As to the available coating methodologies, electroplating is a well-known technique for coating a workpiece with a metal. A workpiece so coated has a surface that is brighter and more corrosion resistant than the substrate to which the coating is applied.

Electroplates are generally applied by immersing a workpiece in a tank containing select chemicals dissolved in water to form a plating bath. The workpiece to be plated is attached to a negative electrical lead, and thus becomes a cathode. The other electrical lead, the positive electrical lead, is in the solution (i.e., the anode). When current is supplied to the plating solution, the negatively charged immersed workpiece attracts the positively charged metal from the solution. This continues as long as current is on, with the layer of deposited metal becoming thicker and thicker as a function among other things, of time.

In chromium plating baths, in addition to a chromium source such as chromic acid, sulfate and fluoride ions may be introduced to act as catalysts. Temperature, current density, and bath composition affect the film characteristics and current efficiency. These parameters are therefore carefully controlled in order to obtain specific deposit properties and plating rates.

As to bath compositions, chromic acid and sulfate are the common ingredients. Generally, chromic-to-sulfate ratios range from 75:1 to 250:1. The specific composition is primarily dependent upon whether the bath is co-catalyzed, e.g., with fluoride, fluorosilicates or fluoroboron. Hexavalent chrome is the source of chromium deposited from such baths, with chromic acid being the main component in the solution make up. During the deposition process, hexavalent chrome is first reduced to trivalent chrome, is next reduced to the unstable divalent state, and further and final reduced to the stable, zero valence, elemental chromium state.

Plating bath temperature is closely related to current density and its effect on brightness and coverage of deposit. Generally, the higher the current density, the higher should be the temperature. An optimum temperature range generally exists for a given concentration of chromic acid. Below or above that range, undesirable dull deposits result. For hard chromium, the range is 120° F. (49° C.) to 150° F. (65.5° C.). Preheating of parts to optimum bath temperature may be needed before they are introduced into the plating tank, and in rare instances, cooling of parts may be required, in order to ensure uniformity of deposit.

At a given solution composition temperature, current density affects cathode efficiency and the deposited chromium's brightness and hardness. Too-high current densities result in burning or roughness of deposition, whereas, at low current densities, the chromium plate may not cover the workpiece unifomrly.

Self-regulating high-speed chromium baths incorporate fluoride complexes such as silicofluoride, in addition to sulfates. Salts of low solubility are used to release the desired anions on a controlled basis. Mixtures containing potassium or sodium silicofluoride and dichromate, for example, regulate the release of fluoride via the common-ion affect. Mixtures of strontium sulfate and chromate regulate the release of sulfate in solution. Consequently, at higher temperatures, the cathode current efficiency increases as a result of the increased solubility of catalysts in this type of bath.

The preferred electroplating bath composition of the subject invention includes deionized water heated to 135-140° F. with the following commercially available constituents: HEEF-25 hard chrome plating solution, which is added to the water to form a concentration of 30-35 oz. chromium per gallon and 0.3-0.35 oz. per gallon sulfuric acid. HEEF-25 is available commercially from Atotech Corp., Rock Hills, S.C.

The activating solution comprises Oakite 90 alkaline cleaner, 8-12 oz. per gallon, heated to 90-105° F.; 35% sulfuric acid, 4 oz. per gallon; and ammonium bifluoride salts at ambient temperature (i.e., 65-80° F.) to form HF in the bath. After activation, any common dish detergent at about 4 oz. per gallon may be used to clean the workpiece.

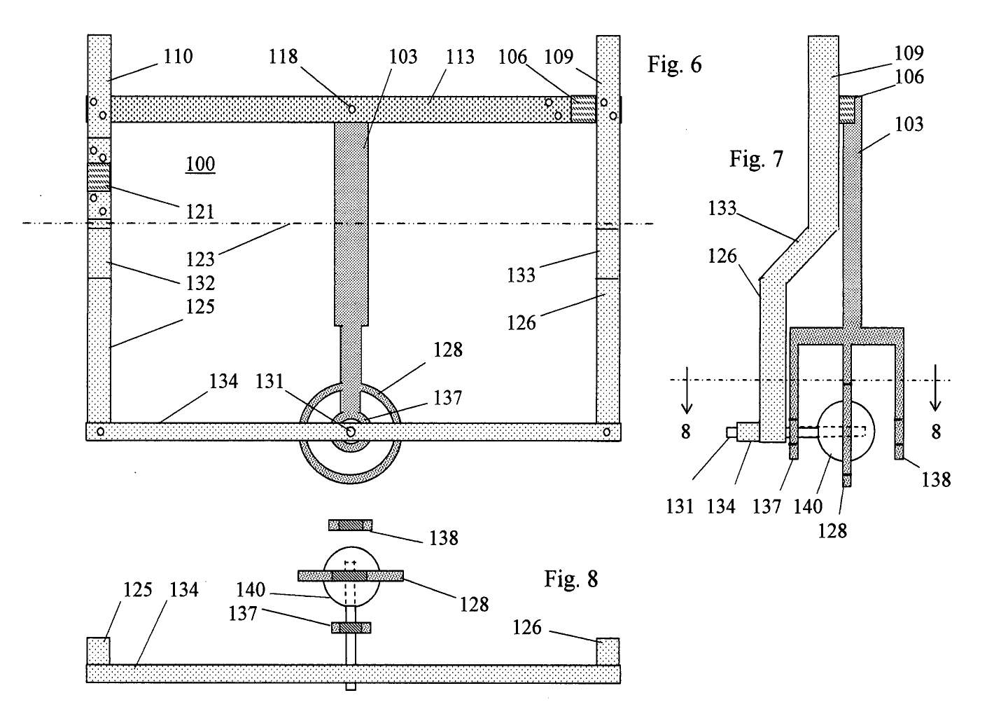

The plating process of the subject invention utilizes conventional electroplating equipment. The equipment preferably includes: a poly-lined steel plating tank with air agitation; quartz heaters providing 5,000 watts; a temperature control unit, including thermostat and thermocouple; a rapid rectifier having a DC 480 3-phase input, 0-9 volt DC output, less than 5% ripple; a steel tank for cleaning process equipped with 2,000-watt electric heaters and temperature control; triple stage cold water rinse tanks; poly acid clean tanks; and, a plating fixture as shown in FIGS. 6-8.

The plating process of the subject invention may be used to plate a selected bearing surface of a cobalt-chrome prosthetic implant serving as a process workpiece. One preferred implant alloy is almost completely cobalt and chromium. Such an alloy may be 30% cobalt, 65% chromium and 6.5% molybdenum with at most a trace amount of iron. The process as described may also perform well for other cobalt-chrome alloys having as much as 10% iron replacing the cobalt and chromium. An excessive percentage of iron results in pitting of the surface during the activation step.

The process has two significant steps. The first step activates the implant surface to prepare it to receive a chromium plate. The second step is the plating step. In this process the implant functions in the activating step as an anode and in the plating step as a cathode.

The implant is placed on one bus of a two-bus bar fixture, see for example FIGS. 6-8. The implant is next wiped with a lint-free rag soaked in a dish detergent solution at room temperature (i.e., between about 65-75 ° F.). The implant is subsequently positioned such that no less than 0.5 in. or more than one inch of spacing is present between it and an element that will function as the cathode. The implant is next rinsed with cold clean running water, and is thereafter submerged in alkaline cleaning solution to cover the implant with about two inches of the cleaning solution. The implant is submerged in the solution for approximately two minutes, while gently agitating the implant by hand. The fixture is then removed and placed in cold, clean running water to remove the cleaning solution.

The activating step occurs next and involves submerging the implant surface in an acid bath to allow about two inches or more of solution to cover the implant. A preferred acid bath includes a 35% sulfuric acid concentration and a dissolved ammonium biflouride salt with an initial concentration of about 4 oz. per gallon of bath solution. It is also possible that sodium biflouride and potassium biflouride will be suitable as an additive to replace the ammonium biflouride. The fixture is anodically activated for approximately 30 seconds at about 3 volts direct current, which should provide a current flow of approximately 2-3 amps per square inch of cathode area.

A relatively wide range of activating step parameter values may well also be suitable. The following table conveniently presents approximations for these values.

Parameter Ranges for Activating Step

| | | | Currently Preferred | |
|---|---|---|---|---|
| Parameter | Acceptable Range | | Value/Range | |
| Sulfuric acid concentration | 20-60% | | 35% | |
| Ammonium biflouride | 2-6 | oz./gal. | 4 | oz./gal. |
| concentration | | | | |
| Activating current density | 1-4 | amp./in.² | 1-4 | amp./in.² |
| Activating duration | 20-40 | sec. | 20-40 | sec. |

The implant is next removed from the activating solution and rinsed with cold clean running water. The implant is then coated with a detergent solution to enhance wetting of the surface during the plating step.

The implant is then submerged with the detergent solution on the surface, in a chrome plating bath comprising a chromic acid sulfate solution. The implant becomes the cathode and is connected to the negative terminal of a DC current source. The DC voltage is preferably set to about 3.2 volts. The DC current is then periodically adjusted upward at a rate of approximately 0.1 volt every 10 seconds, until a voltage of approximately 4-5 volts, and preferably 4.5 volts, is achieved.

A relatively wide range of plating step parameter values may well also be suitable. The following table conveniently presents approximations for these values.

Parameter Ranges for Plating Step

| | | | Currently Preferred | |
|---|---|---|---|---|
| Parameter | Acceptable Range | | Range/Value | |
| Chromic acid | 20-50 | oz. Cr/gal. | 30-35 | oz. Cr/gal. |
| concentration | | | | |
| Sulfuric acid | .2-.5 | oz. H₂SO₄/gal. | 0.3-0.35 | H₂SO₄/gal. |
| concentration | | | | |
| Initial DC voltage | 3-3.5 | v. | 3.2 | v. |
| Voltage step | .05-0.2 | v./interval | 0.1 | v./interval |
| increase | | | | |
| Voltage step interval | 5-10 | sec. | 10 | sec. |
| Maximum voltage | 4-6 | v. | 4.5 | v. |
| Planting duration | 20-60 | min. total | 40-50 | min. total |

The amperage will be noted and calculated to be within a range of about 1-3 amp., and a most preferred range of about 1.5 to 2.5 amp. per square inch of cathode area. A plating rate (i.e., rate of deposition) of approximately 0.0001 in. every 6 minutes results, requiring a plating time of approximately 48 minutes to deposit a chromium coating of 0.0008 inches minimum thickness. At the end of the plating run, the fixture is removed from the plating bath, and rinsed in a triple stage return rinse tank.

The preferred parameters have been tested and are known to provide a suitable chromium layer. However, the relatively wide range of values specified for several of these parameters is likely to also successfully plate workpieces.

The implant is next removed from the plating bath and forced-air dried. The implant removed from the fixture. The implant is hot and cold water rinsed no less than three separate times to remove any residual chrome solution. The implant is then examined by the operator for any stains or discoloration on any of the internal or external surfaces of the implant. Soaking in clean, hot water and wiping with a lint free cloth will usually remove any stain or discoloration. All cleaning operations should be performed within five minutes of removal from the plating solution.

The implant is next examined for thickness, uniformity of coating, and cleanliness. Thereafter, the implant is lapped, polished, and inspected for uniformity of coating and acceptable surface finish, and packaged.

FIGS. 6-8 show a fixture or frame 100 suitable for supporting in a preselected spatial relationship during the previously described process, a spherical implant workpiece 140 and a plating anode 103. Frame 100 is used to support workpiece 140 for both the activating and the plating steps. A representative fluid level for the activating and plating solutions is shown at 123.

FIGS. 6 and 7 are respectively front and side views of frame 100. FIG. 7 is in projective alignment with FIG. 6. FIG. 8 is a section view of FIG. 7, and is in projective alignment with FIG. 6. Mild steel bolts and nuts as at 118 form fasteners that mechanically and electrically connect bars formed of copper, lead, and aluminum to form frame 100.

A first copper electrode bar 110 oriented generally vertically serves as a first electrical connection and is used to support frame 100 in the activating and plating paths. A first end of a copper bar 113 is fastened to bar 110 and extends horizontally to form an upper member of frame 100. An insulating bar 106 is riveted to a second end of copper bar 113 and extends generally horizontally.

A second copper electrode bar 109 serves as a second electrical connection, and is fastened to the insulating bar 106 so that bar 109 is electrically insulated from bar 113. Bar 109 extends vertically downwards from insulating bar 106. An aluminum bar 126 is bolted to the lower end of bar 109 and extends downwardly to a bottom edge of frame 100.

An insulating bar 121 is bolted to the lower end of bar 110 and extends downwardly therefrom. An aluminum bar 125 is bolted to the lower end of insulating bar 121 and extends downwardly to the bottom edge of frame 100.

Parts of the frame 100 that are immersed in the activating and plating solutions should be formed of aluminum to provide good electrical conductivity and resist corrosion. Bars 109, 110, and 113 are made of copper to provide good electrical conductivity but cannot be immersed in the activating and plating solutions because the copper will rapidly corrode.

The lower ends of aluminum bars 125 and 126 should be in approximate horizontal alignment. An aluminum workpiece support bar 134 is bolted to the lower ends of bars 125 and 126 and extends horizontally between bars 125 and 126. Bar 134 supports a pin 131 on which workpiece 140 is supported in a spaced relation to bar 134 approximately midway between bars 125 and 126. For a workpiece 140 of 28-32 mm. dia., the height of frame 100 between bars 113 and 134 may be approximately 28 cm. The width may be approximately 45 cm.

Bar 113 supports an anode element 103. An upper end of element 103 is bolted to bar 113 approximately midway between bars 125 and 126. Element 103 extends vertically downwardly from bar 113. Bar 103 is preferably formed of an alloy comprising approximately 4-6% antimony with the remainder lead. Lead must be used because other common metals are either rapidly corroded by the baths or form a resistive film that reduces flow of electricity. The antimony is added to improve the mechanical strength of the lead.

The lower end of bar 103 has a special shape to at least partially surround workpiece 140. In the version shown in FIGS. 6-8 for use with a spherical workpiece 140, the lower end of bar 103 comprises three concentric, spaced rings 128, 137, and 138 that at least partially surround workpiece 140. The lead-antimony alloy is relatively soft, so that one of the arms supporting rings 137 and 138 may be bent to allow workpiece to be placed within ring 128 and then bent back into the shape shown.

To space rings 128, 137, and 138 properly from bar 134, bars 125 and 126 may require offset bends 132 and 133 as shown. Other spacers and offsets in other of the various bars may be required to establish needed clearances and rectilinear alignments. All of these details are well within the ability of those having skill in these plating arts.

To insure relatively uniform plating thickness on workpiece 140, during the plating step, all parts of workpiece 140 should be spaced within 12-25 mm. from rings 128, 137, and 138. That is, every part of the workpiece surface should be within 25 mm. of some part of rings 128, 137, and 138, and no area of workpiece 140 should be closer than 12 mm. of rings 128, 137, and 138.

Since many possible embodiments may be made of the present invention without departing from the scope thereof, it is to be understood that all matter herein set forth or shown in the accompanying drawings is to be interpreted in the illustrative and not limiting sense.

1. An electrolytic process for preparing a surface for plating a chromium layer thereon, the steps comprising:
a) providing a workpiece carrying the surface to be prepared, said workpiece formed of an alloy comprised at least in part of cobalt and chromium; and then
b) activating at least a selected area of the surface by submerging the selected area in an aqueous solution including sulfuric acid and a dissolved biflouride salt.

2. The process of claim 1, wherein the activating step includes placing the selected area in an aqueous solution having ammonium biflouride as the biflouride salt.

3. The process of claim 1, further adapted for plating the chromium layer onto the surface, and comprising the steps of:
a) submerging the selected area in a chromic acid sulfate plating bath; and then
b) plating chromium on the selected area by applying a negative DC plating voltage between the workpiece and an anode in the plating bath.

4. The process of claim 3, wherein the plating step comprises applying a DC plating voltage sufficient to create an initial current density on the selected area of from about 1 to about 4 amp. per sq. in.

5. The process of claim 4, wherein the plating step includes the further step of increasing the plating voltage over time.

6. The process of claim 5, wherein the plating step includes increasing the plating voltage by an amount within the range of about 0.05 v. to about 0.2 v. about every 5 to 10 sec.

7. The process of claim 6, wherein the plating step includes increasing the plating voltage to a maximum of about 4 to about 6 v.

8. The process of claim 7, wherein the plating step includes increasing the plating voltage by about 0.1 v. every about 10 sec.

9. The process of claim 6, wherein the plating step includes increasing the plating voltage to a maximum of about 4.5 v.

10. The process of claim 9, wherein the plating step includes increasing the plating voltage by about 0.1 v. every about 10 sec.

11. The process of claim 2, wherein the activating step includes applying a positive DC voltage between the workpiece and a cathode in the aqueous solution.

12. The process of claim 11, wherein the activating step comprises applying DC voltage in the range of about 2 v. to about 4 v. for from about 20 to about 40 sec.

13. The process of claim 11, wherein the activating step comprises selecting a voltage providing a current density of about 1 to about 4 amp. per sq. in. on the selected area.

14. The process of claim 13, wherein the activating step further includes the step of placing the workpiece in a solution having a sulfuric acid concentration of about 20% to about 60%.

15. The process of claim 14, wherein the activating step further includes the step of placing the workpiece in a solution whose ammonium biflouride crystal concentration is about 2 to about 6 oz. per gal.

16. The process of claim 15, wherein the activating step further includes the step of placing the workpiece in a solution having a sulfuric acid concentration of about 35%.

17. The process of claim 16, wherein the activating step further includes the step of placing the workpiece in a solution having an ammonium biflouride crystal concentration of about 4 oz. per gal.

18. The process of claim 15 further adapted for plating the chromium layer onto the surface, and comprising the steps of:
a) submerging the workpiece in a chromic acid sulfate plating bath; and then
b) plating chromium on the selected area by applying a negative DC plating voltage between the workpiece and an anode in the plating bath.

19. The process of claim 18, wherein the plating step comprises applying a DC plating voltage sufficient to create an initial current density on the selected area of from about 1 to about 4 amp. per sq. in.

20. The process of claim 19, wherein the plating step includes the further step of increasing the plating voltage over time.

21. The process of claim 20, wherein the plating step includes increasing the plating voltage by an amount within the range of about 0.05 v. to about 0.2 v. about every 5 to 10 sec.

22. The process of claim 21, wherein the plating step includes increasing the plating voltage to a maximum of about 4 to about 6 v.

23. The process of claim 22, wherein the plating step includes increasing the plating voltage by about 0.1 v. every about 10 sec.

24. The process of claim 22, wherein the depositing step includes increasing the plating voltage to a maximum of about 4.5 v.

25. The process of claim 24, wherein the depositing step includes increasing the plating voltage by about 0.1 v. every about 10 sec.

26. The process of claim 1, wherein the activating step further includes the step of placing the workpiece in a solution having a sulfuric acid concentration of about 20% to about 60%.

27. The process of claim 26, wherein the activating step further includes the step of placing the workpiece in a solution having an ammonium biflouride crystal concentration of about 2 to about 6 oz. per gal.

28. The process of claim 1, wherein the activating step further includes the step of placing the workpiece in a solution having a sulfuric acid concentration of about 35%.

29. The process of claim 28, wherein the activating step includes applying a positive DC voltage between the workpiece and a cathode in the aqueous solution.

30. The process of claim 29, wherein the activating step comprises applying DC voltage in the range of about 2 v. to about 4 v. for from about 20 to about 40 sec.

31. The process of claim 29, wherein the activating step comprises selecting a voltage providing a current density of about 1 to about 4 amp. per sq. in. on the selected area.

32. The process of claim 28, wherein the workpiece-providing step includes providing a workpiece formed almost completely of cobalt and chromium with at most, trace amounts of iron.

33. The process of claim 32, wherein the workpiece-providing step includes providing a workpiece comprising approximately 30% cobalt and 65% chromium.

34. The process of claim 28, wherein the workpiece-providing step includes providing a workpiece formed almost completely of cobalt and chromium with not more than about 10% of iron.
